(19) United States
(12) Patent Application Publication
(10) Pub. No.: US 2017/0000482 A1
(43) Pub. Date: Jan. 5, 2017

(54) SURGICAL INSTRUMENT FOR FITTING AN OSTEOSYNTHESIS CLIP

(71) Applicant: ADSM, Saint-Maurice -de-Gourdans (FR)

(72) Inventors: Christophe AVEROUS, Mittelhausbergen (FR); Gualter VAZ, Lyon (FR); Frédéric LEIBER-WACKENHEIM, Strasbourg (FR); Christophe CERMOLACCE, Marseille (FR); Patrice DETERME, Toulouse (FR); Stéphane GUILLO, Bordeaux (FR); Hubert ROCHER, Pessac (FR); Christophe ROY, Chatuzange le Goubet (FR); Jean-Charles ROUSSOULY, Villeurbanne (FR); Arnaud FIQUET, Caluire et Cuire (FR); Bertrand SEUTIN, La Batie Rolland (FR)

(73) Assignee: ADSM, Saint-Maurice-de-Gourdans (FR)

(21) Appl. No.: 15/113,025

(22) PCT Filed: Jan. 19, 2015
(86) PCT No.: PCT/FR2015/050119

§ 371 Date: Jul. 20, 2016

(30) Foreign Application Priority Data
Jan. 20, 2014 (FR) 14/50418

(51) Int. Cl. A61B 17/068; A61B 17/064

(52) U.S. Cl. CPC A61B 17/0682; A61B 17/0642; A61B 2017/0645

(57) ABSTRACT

A surgical instrument for fitting a U-shaped osteosynthesis clip having two side bars linked together at the proximal ends by a transverse bar, the transverse bar and the proximal portions forming a proximal region of the clip, the instrument including: at least one recess intended for provisionally receiving the proximal region of the osteosynthesis clip, the distal ends of the two side bars projecting from the instrument; at least blocking means capable of preventing the translation movement of the transverse bar in the proximal and distal directions; at least anti-rotation means preventing the clip from rotating about the transverse bar; and spacing means capable of engaging with the proximal region in order to increase the distance between the distal ends of the two side bars. The invention also relates to a kit including such an instrument and to a clip intended for being received in such an instrument.

The present invention relates to an instrument for fitting an osteosynthesis clip, for example to carry out an osteotomy, an arthrodesis between two fragments of bone, or even to attach soft tissue to bone.

There are numerous surgical clips in existence for effecting bone repairs. Some of these clips are in the overall shape of an inverted U, the distal ends of the lateral legs of the U, or anchor legs, being intended to be inserted into two different fragments of bone in order to pull these together with compression in order to obtain bone fusion for example. The anchor legs are joined together by a central beam that forms the solid connection between these anchor legs and is known hereinafter as a crossbar.

In this application, the distal end of a device, such as an instrument or a clip, is to be understood to mean the end furthest away from the hand of the surgeon and the proximal end is to be understood as meaning the end closest to the hand of the surgeon. Likewise, in this application, the “distal direction” is to be understood to mean the direction of impaction of the clip and the “proximal direction” is to be understood to mean the opposite direction to the direction of impaction. In the present application, the longitudinal direction of a device is to be understood as meaning the direction aligned with the proximal-distal axis, and the transverse direction is to be understood as being the direction perpendicular to the longitudinal direction within the plane of the device.

U-shaped clips need to be able to offer a certain degree of elasticity in order to encourage the lateral legs and the crossbar to bend. Indeed generally, an osteosynthesis clip is elastically deformable and is able to adopt a configuration at rest, closed, in which each lateral leg makes an angle a with the crossbar, and an open configuration under stress loading, in which each lateral leg forms an angle β with the crossbar, the angle p being greater than the angle a so that the crossbars spread apart as the clip passes from its closed configuration to its open configuration. Thus, when the clip is in the open configuration, the distal ends of the two lateral legs are separated by a distance that is greater than the distance between these two same distal ends when the clip is in the rest configuration.

An osteosynthesis clip is intended to be implanted in its open configuration, under stress, into two elements of bone that are to be pulled closer together. Thus, once implanted, it has a natural tendency to return to its rest configuration, under no stress, each lateral leg thus applying a compression force that tends to move the element of bone in which it is implanted toward the other element of bone in which the other lateral leg is implanted. The osteosynthesis clip thus allows elements of bone to be held postoperatively in a normal physiological position and compressed for the time it takes the bone to consolidate.

There is therefore a need for an instrument that is easy to handle, capable first of all of accepting the clip in its rest configuration and then of placing it in its configuration under stress loading and finally of implanting it, in other words of inserting it into the fragments of bone that are to be pulled together.

The present invention seeks to overcome this requirement by proposing a surgical instrument for the fixing of an osteosynthesis clip in the overall shape of a U that is to be brought into a configuration under stress loading at the time of implantation into the fragments of bone.

The present invention relates to a surgical instrument for implanting a U-shaped osteosynthesis clip having two lateral legs joined together at their respective proximal ends by a crossbar, said crossbar and the proximal portions of said two lateral legs forming a proximal region of said clip, said instrument comprising:
at least one housing intended to temporarily receive said proximal region of said clip, the distal ends of said two lateral legs protruding out of said instrument,
at least blocking means able to prevent translational movement of said crossbar in the proximal and distal directions,
at least rotation-proofing means preventing the clip from rotating about its crossbar,
at least spreader means able to collaborate with said proximal region of said clip in order to increase the distance between the distal ends of the two lateral legs, said spreader means comprising a stop-forming structure located inside the U formed by the clip, in the proximal region thereof, said structure bearing against the proximal portion of each of said lateral legs, said structure being able to increase in size in the transverse direction in order to spread said distal ends of the two lateral legs apart, said structure comprising two adjacent lugs able to be moved relative to one another in the transverse direction, and an insert able to be accommodated temporarily between said two lugs,
said instrument comprising at least one pair of tongs comprising two arms joined together at their proximal ends by a joining bridge, the two arms being parallel when not subjected to any stress loading, said two arms being able to be brought closer together and to form between them a non-zero angle under the effect of pressure applied to each of them toward the other, each of said two lugs being situated on one of said two arms, facing one another,
characterized in that said insert is a rod the proximal end of which passes through the center of said joining bridge and the distal end of which is accommodated between said two lugs.

The instrument according to the invention allows a U-shaped osteosynthesis clip under stress loading to be placed with ease. As will become apparent from the description which follows, the instrument according to the invention is particularly easy to handle.

Said spreader means comprise a stop-forming structure arranged inside the U formed by the clip, in the proximal region thereof when the latter is housed in the housing of the instrument provided for that purpose, said structure bearing against the proximal portion of each of said lateral legs, said structure being able to increase in size in the transverse direction in order to spread said distal ends of the two lateral legs apart. Said structure thus comprises two adjacent lugs that can be moved relative to one another in the transverse direction, and an insert able to be accommodated temporarily between said two lugs. Thus, when the insert is not present between the two lugs and the two lugs are in contact with one another, the dimension of said structure in the transverse direction is made up of the dimensions of the two lugs placed side by side. This dimension corresponds for example to the distance between the proximal portions of the two lateral legs of the U-shaped clip when the U-shaped clip is at rest. By contrast, when the insert is placed between the two lugs, the latter spread away from one another and the dimension of said structure in the transverse direction is then made up of the dimensions of the two lugs plus the dimension of the insert. The dimension of the structure is therefore increased and corresponds to a distance between the distal ends of the two lateral legs of the U-shaped clip that is increased: the U-shaped clip is then under stress loading.

The instrument comprises at least one pair of tongs comprising two arms joined together at their proximal ends by a joining bridge, the two arms being parallel when not subjected to any stress loading, said two arms being able to be brought closer together and form between them a non-zero angle under the effect of pressure applied to each of them toward the other, each of said two lugs being situated on one of said two arms, facing one another. The two arms of the instrument thus form a kind of sugar tongs or U-shaped tongs with elastic return, and it is easy to bring the lugs closer together by pressing against the two arms, like for closing the tongs.

Said insert is a rod the proximal end of which passes through the center of said joining bridge and the distal end of which is accommodated between said two lugs. When the distal end of the rod is accommodated between the two lugs, the dimension of the stop-forming structure in the transverse direction is increased in comparison with the situation in which the two lugs are in contact with one another. The clip present in the housing is therefore placed in its stressed configuration and the distal ends of its lateral legs are spread apart in comparison with the rest configuration of the clip.

In one embodiment, at least part of said spreader means is removable. For example, the insert is removable and constitutes a separate piece of said tongs. For example, the rod is removable and constitutes a separate piece of said tongs.

In one embodiment, the instrument further comprises, on a distal face, a transverse groove able to accommodate said crossbar. Thus, when the clip under stress loading has begun to be inserted into the fragments of bone that are to be pulled together, it is removed from the housing of the instrument. The crossbar of the clip is therefore slid into the transverse groove situated on one distal face of the instrument and it is possible to push on the instrument in order to continue to embed the clip in the fragments of bone.

In one embodiment, the instrument further comprises, on a proximal face, a bearing surface able to receive distal pressure. Thus, when the crossbar of the clip half inserted into the fragments of bone is housed in the transverse groove, the surgeon can hammer the surface situated on the proximal face of the instrument in order to embed the clip in the fragments of bone.

In one embodiment, said housing comprises, preferably consists of, an open groove formed on one face of said instrument. Such an open groove allows the clip to be loaded onto and unloaded from the instrument with particular ease.

In one embodiment, said blocking means comprise a transverse slot comprising a proximal face and a distal face, said transverse slot being able to receive the crossbar of the clip. Engagement of the crossbar of the clip in the transverse slot blocks any movement of the clip in the distal direction or the proximal direction. The clip can therefore be impacted in complete safety.

The instrument according to the invention comprises rotation proofing means preventing the clip from rotating about its crossbar when the proximal region thereof is housed in said housing. In one embodiment, said rotation proofing means comprise two longitudinal slots able to receive the proximal portions of the lateral legs of the clip. Because the proximal portions of the lateral legs are held in the longitudinal slots, the clip is unable to rotate about the axis defined by the crossbar. Thus, the clip is held in a determined plane, particularly in the plane corresponding to the direction of impaction of the clip into the fragments of bone that are to be pulled together. In one embodiment, the rotation proofing means may comprise, for example in addition to the two longitudinal slots able to accept the proximal portions of the lateral legs of the clip, at least one flat situated at one distal end of said rod, said at least one flat coming to bear against said crossbar when the proximal region of said clip is received in said housing and the distal end of said rod is accommodated between said two lugs. For example, said rotation proofing means may comprise two flats situated at one distal end of said rod, each flat coming to bear against said crossbar on each side thereof when the proximal region of said clip is received in said housing and the distal end of said rod is accommodated between said two lugs.

In one embodiment, the instrument comprises information means indicating the center distance of the clip intended to be received in said instrument when this clip is under stress loading. The center distance of a U-shaped clip corresponds to the distance between the respective distal ends of the two lateral legs. For example, these information means may be transverse holes made in the instrument. For example, said information means comprise two transverse holes formed in said instrument, the center distance between said two holes corresponding to the center distance of the clip under stress loading. Thus, before the clip is installed, the surgeon may, in advance, create in the fragments of bone the holes that are intended to accept the distal ends of the two lateral bars, based on information given by the relative position of the transverse holes of the instrument, particularly the distance from one transverse hole to the other.

Another aspect of the present invention is a kit comprising an instrument as described hereinabove and at least one U-shaped osteosynthesis clip having two lateral legs joined together at their respective proximal ends by a crossbar.

The advantages of the present invention will become more clearly apparent from the description which follows and from the attached drawings in which:

FIG. 1 is a front-on view of a U-shaped clip that can be used with the instrument according to the invention, in its rest configuration,

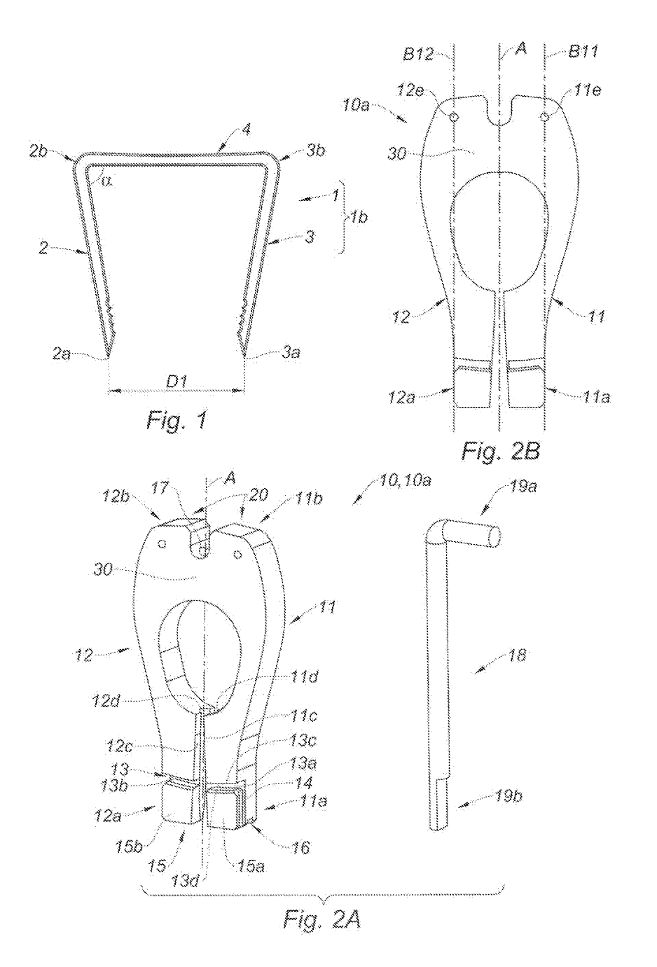

FIG. 2A is a perspective view of one form of embodiment of the instrument according to the invention,

FIG. 2B is a front-on view of the tongs of the instrument of FIG. 2A,

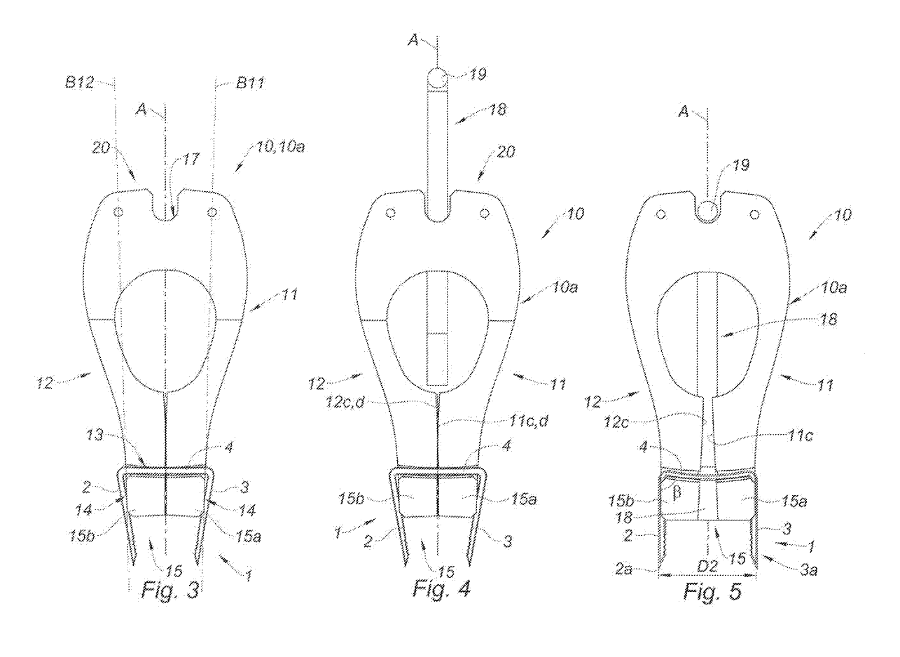

FIG. 3 is a front-on view of the instrument according to the invention with the clip of FIG. 1 loaded onto the instrument, in the rest configuration,

FIG. 4 is a front-on view of the instrument according to the invention with the clip of FIG. 1 loaded onto the instrument, in the rest configuration, before the start of placement of the insert,

FIG. 5 is a front-on view of the instrument according to the invention with the clip of FIG. 1 loaded onto the instrument, with the insert in place and the clip in the stress-loaded configuration,

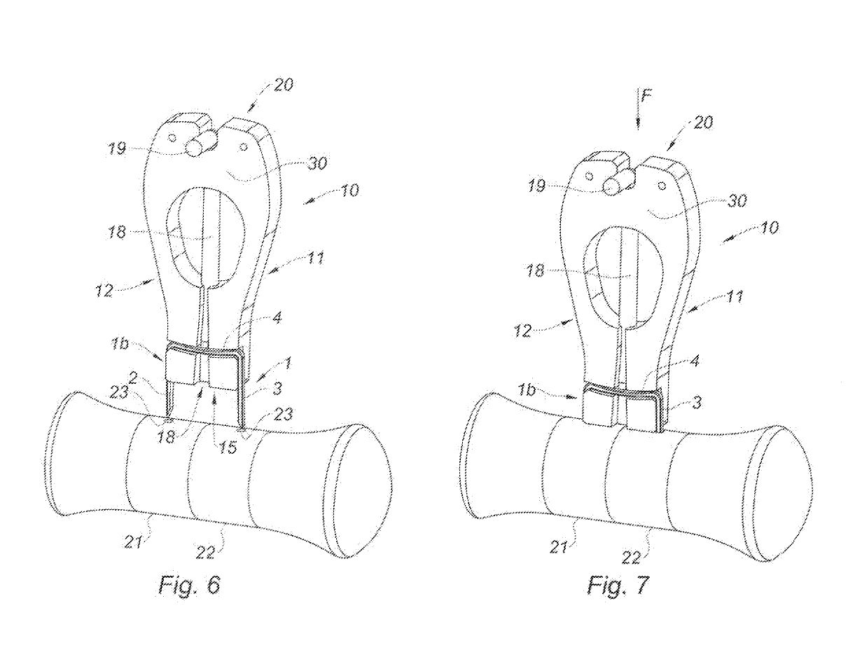

FIG. 6 is a perspective view of the first step in the placement of the clip of FIG. 1 in fragments of bone using the instrument of FIG. 2A,

FIG. 7 is a perspective view of the second step of the placement of the clip of FIG. 1 using the instrument of FIG. 2A, the clip being half inserted into the fragments of bone,

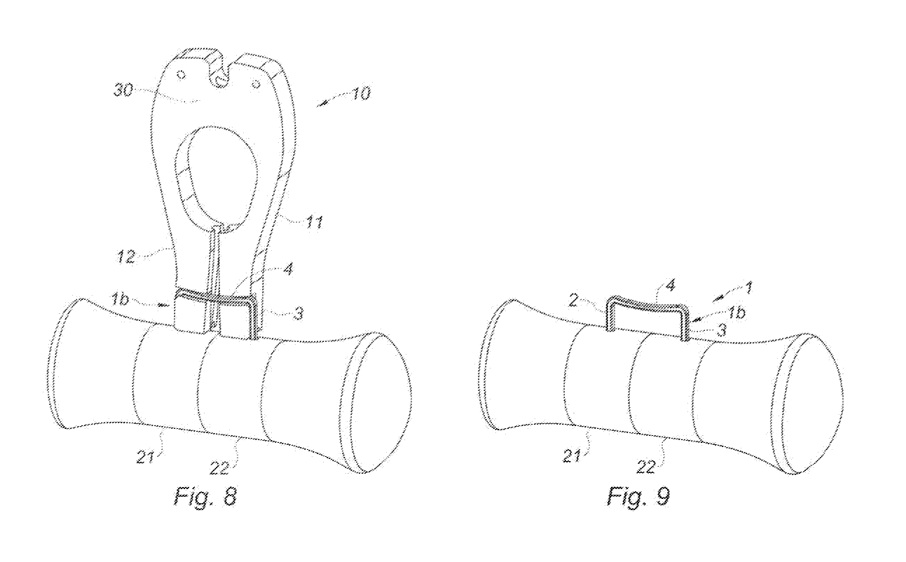

FIG. 8 is a perspective view of a third step in the placement of the clip of FIG. 1 using the instrument of FIG. 2A, the clip being half inserted into the fragments of bone, the insert having been removed,

FIG. 9 is a perspective view of the clip of FIG. 1 half inserted into the fragments of bone,

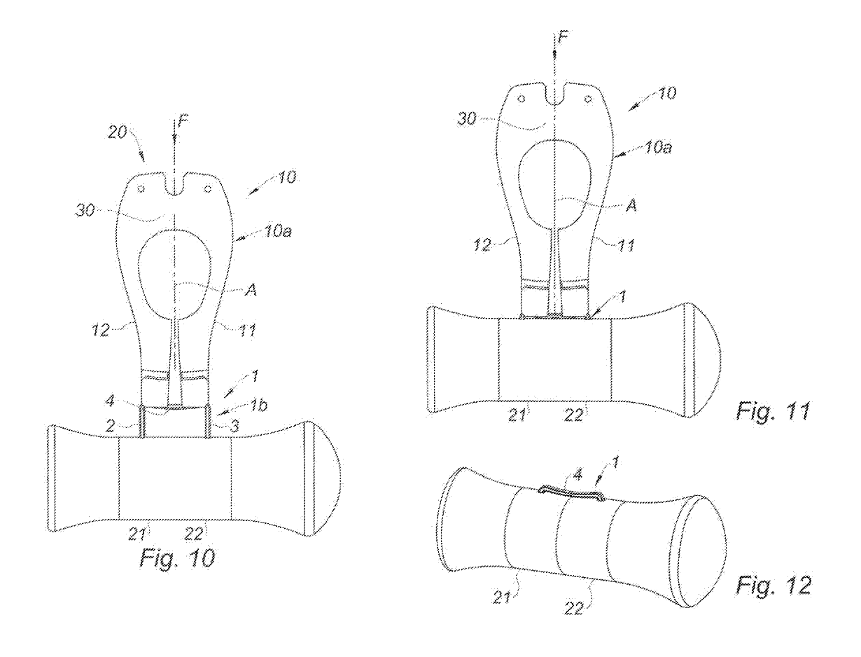

FIG. 10 is a front-on view of a fourth step in the placement of the clip of FIG. 1 using the instrument of FIG. 2A, the clip being half inserted into the fragments of bone, the instrument according to the invention being used to continue to insert the clip into the fragments of bone,

FIG. 11 is a front-on view of a fifth embodiment of the placement of the clip of FIG. 1 using the instrument of FIG. 2A, the clip being completely inserted into the fragments of bone,

FIG. 12 is a perspective view of the clip of FIG. 1 fully inserted into the fragments of bone,

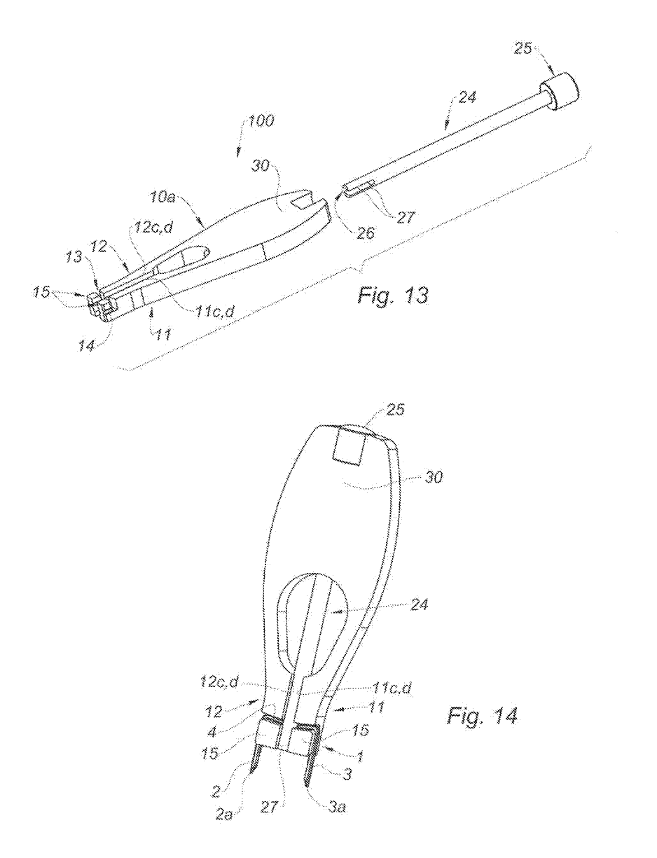

FIG. 13 is a perspective view of a second embodiment of the instrument according to the invention with the insert not mounted on the instrument tongs,

FIG. 14 is a perspective view of the instrument of FIG. 13, with the insert mounted on the tongs.

Reference is made to FIG. 1 which depicts a clip 1 that can be used with the instrument 10 according to the invention (see FIG. 2A), for example for pulling together two fragments of bone (21, 22) (see FIG. 6). The fragments of bone may be fragments of bone in the foot or in the hand, or even two adjacent vertebrae that need pulling together or even any other fragmented bone. The clip 1 has the overall shape of an inverted U and comprises two lateral legs (2, 3) corresponding to the vertical legs of the U. Each lateral leg (2, 3) comprises a distal end (2a, 3a) intended to be inserted into a fragment of bone. Each lateral leg (2, 3) also has a proximal end (2b, 3b) situated at the opposite end to the distal end (2a, 3a).

The clip 1 further comprises a crossbar 4 corresponding to the horizontal bar of the U and connecting the respective proximal ends (2b, 3b) of the two lateral legs (2, 3) together. As can be seen in the figures, the two lateral legs (2, 3) and the crossbar 4 are situated in one and the same plane, the plane of the U. The crossbar may for example be of square or rectangular cross section.

The crossbar 4 and the proximal portions of the two lateral legs (2, 3) form a proximal region lb of the clip 1, as shown in FIG. 1.

As will be apparent from FIG. 5, the clip 1 is elastically deformable and is able to adopt a closed rest configuration, depicted in FIG. 1, in which each lateral leg (2, 3) makes an angle a with the crossbar 4, and an open stress-loaded configuration, depicted in FIG. 5, in which each lateral leg (2, 3) makes an angle 13 with the crossbar 4, the angle 13 being greater than the angle a so that the distal ends (2a, 3a) of the lateral legs (2, 3) spread apart as the clip 1 passes from its closed configuration into its open configuration.

Thus, in the example depicted, the angle a is an acute angle so that the distal ends (2a, 3a) of the lateral legs (2, 3) are directed toward the inside of the U when the clip 1 is in the rest configuration (FIG. 1) when no stress loading is applied to the lateral legs (2, 3). In this same example, the angle is a substantially right angled angle so that, in the open stress-loaded configuration of the clip 1, which is depicted in FIG. 5, the two lateral legs (2, 3) are substantially parallel to one another. The distance D2 separating the distal ends (2a, 3a) of the lateral legs (2, 3) in the stress-loaded configuration (FIG. 5) of the clip 1 is therefore greater than the distance D1 separating the distal ends (2a, 3a) of the lateral legs (2, 3) in the rest configuration (FIG. 1) of said clip 1.

However, in embodiments that have not been depicted, the angles a and 13 could have different values from those mentioned hereinabove.

The clip 1 may be made of a biocompatible material that allows elastic deformation of the clip, for example a biocompatible material selected from stainless steel, chrome/cobalt alloys, polylactic acid, polyetheretherketone, titanium and alloys thereof, and mixtures of these.

Reference is made to FIG. 2A which depicts an instrument 10 according to the invention for implanting a clip 1 of FIG. 1 into fragments of bone with a view to pulling said fragments closer together.

The instrument 10 as depicted in FIG. 2A comprises a pair of tongs 10a and a rod 18 forming an insert.

The pair of tongs 10a comprises a first arm 11 and a second arm 12 which are symmetrical with respect to a longitudinal axis A aligned with the proximal-distal direction in the example depicted, joined together by their respective proximal ends (11b, 12b) by a joining bridge 30, their respective distal ends (11a, 12a) remaining free. The longitudinal axis A passes through the center of the joining bridge 30. When the tongs 10a are in the rest position, which means to say when no stress loading is applied to the two arms (11, 12) as depicted in FIGS. 2A and 2B, said two arms (11, 12) are mutually parallel. FIG. 2B depicts the longitudinal axis B11 of the arm 11 and the longitudinal axis B12 of the arm 12. These two axes are mutually parallel when the tongs 10a are in the rest position. The two arms (11, 12) can be moved closer together under the effect of pressure applied to each of them, particularly at its distal end (11a, 12a) toward the other leg or toward the longitudinal axis A, as in the case of sugar tongs. The arms (11, 12) between them thus form a non-zero angle (see FIG. 3): as can be seen from FIG. 3, the axes B11 and B12 are no longer parallel to one another when the two arms (11, 12) are in this stress-loaded position.

The tongs 10a of the instrument 10 are equipped on their front face with a transverse slot 13 formed of a first slot portion 13a situated on the front face of the distal end of the first arm 11 and of a second slot portion 13b situated on the front face of the distal end of the second arm 12, said two portions (13a, 13b) being in the transverse continuation one of the other, so that when pressure is applied to said distal ends in order to bring the arms (11, 12) closer together, the two portions then merely form a single continuous slot 13 (see FIG. 3). In the example depicted, the transverse slot 13 is arcuate so as to adapt to the deformation of the crossbar 4 of the clip 1 when the latter is in its stress-loaded configuration. In other embodiments, the shape of the slot may differ so as to conform to the shape of the crossbar when the clip is under stress loading.

With reference to FIGS. 2A and 2B, the transverse slot 13 comprises a proximal face 13c and a distal face 13d. The distal ends (11a, 12a) of the arms (11, 12) are symmetrical with respect to the longitudinal axis A. The transverse slot 13 is extended at each of its ends by a longitudinal slot 14 extending in the distal direction.

As will become apparent in the description that follows, the transverse slot 13 and the two longitudinal slots 14 form a U-shaped housing able to accept the proximal region 1b of the clip 1 of FIG. 1 in its rest position, when the distal ends of the arms (11, 12) are brought closer together. The open nature of the transverse 13 and longitudinal 14 slots allows the clip 1 to be loaded onto and unloaded from the tongs 10a with ease. As will become apparent from the description that follows, the proximal face 13c and the distal face 13d form blocking means able to prevent translational movement of the crossbar of the clip 1 in the proximal and distal directions when this clip is mounted on the instrument 10. Likewise, it will become apparent from the description that follows that the longitudinal slots 14 form rotation proofing means preventing the clip 1 from rotating about its crossbar 4 when the clip 1 is mounted on the tongs 10a of the instrument 10.

The distal face 13d of the transverse groove 13 and the longitudinal grooves 14 define a stop-forming structure 15 formed on the front face of the tongs 10a of the instrument 10. The stop-forming structure 15 is formed of a first lug 15a situated on the front face of the distal end of the first arm 11 and of a second lug 15b situated on the front face of the distal end of the second arm 12. The two lugs (15a, 15b) are adjacent and in the continuation of one another in the transverse direction which means that when there is pressure applied to said distal ends with a view to bringing the legs closer together, the two lugs (15a, 15b) are in contact with one another and form just one single continuous stop-forming structure 15 (see FIG. 3).

The distal face of the tongs 10a of the instrument 10 further comprises a transverse groove 16, just one end of which is visible in FIG. 2A. Like the transverse slot 13, the transverse groove 16 is formed of a first groove portion situated on the distal face of the distal end of the first arm 11 and of a second groove portion situated on the distal face of the distal end of the second arm 12, said two portions being in the continuation of one another, so that when pressure is applied to said distal ends in order to bring the arms (11, 12) closer together, the two portions form just one single continuous groove 16.

The distal end 11a of the first arm 11 comprises on its face 11c situated facing the distal end 12a of the second arm 12 a partially semicylindrical longitudinal recess 11d extending as far as the distal end of the first lug 15a. In the same way, the distal end 12a of the second arm 12 comprises, on its face 12c situated facing the distal end 11a of the first arm 11, a partially semicylindrical longitudinal recess 12d extending as far as the distal end of the second lug 15b. The two partially semicylindrical longitudinal recesses (11d, 12d) face one another so that when pressure is applied to said distal ends in order to move the arms closer together, the two partially semicylindrical recesses together form a longitudinal recess which in the example depicted is substantially conical in shape.

Each arm (11, 12) also, in the proximal region of its front face, has a through-hole (11e, 12e), see FIG. 2B, situated on its longitudinal axis (B11, B12): the center distance between the two transverse holes (11e, 12e) when no pressure is being applied to the arms (11, 12) of the tongs 10a, corresponds to the center distance between the distal ends (2a, 3a) of the clip 1 under stress loading (see also FIG. 5). Thus, the position of the arms (11, 12) of the tongs 10a, namely the fact that these are mutually parallel, is the same both when the tongs 10a are not subjected to any stress loading, as shown in FIGS. 2A and 2B, and when the clip 1 is mounted on this pair of tongs 10a and the rod 18 that forms an insert is also mounted on the pair of tongs 10a, as shown in FIG. 5. The through-holes (11e, 12e) thus make it possible to inform the surgeon as to how much distance to provide between the holes 23 (see FIG. 6) to be made in the fragments of bone (21, 22 prior to implanting the clip 1. The through-holes (11e, 12e) thus act as a drilling template for making the holes in the fragments of bone.

The joining bridge 30 is equipped at its center with an axial hole 17 (see FIG. 2A).

Still with reference to FIG. 2A, the instrument 10 further comprises a removable insert in the form of a rod 18 equipped at its proximal end with a transverse projection 19a for grasping and at its distal end with a flat 19b. As will become apparent from the description that follows, the flat 19b contributes to keeping the clip 1 in the plane of the tongs 10a, or in other words contributes to preventing the clip 1 from rotating about its crossbar 4 when said clip 1 is being impacted. The rod 18 is dimensioned in such a way as to be able to pass through the axial hole 17 via its distal end. When pressure is applied to the distal ends of the arms in order to bring said arms closer together, the conical recess mentioned hereinabove and formed by the two partially semicylindrical recesses offers a cone inlet of a diameter greater than the diameter of the rod 18, so that when the rod 18 is brought to face the two partially semicylindrical longitudinal recesses (11d, 12d) it spreads the two distal ends of the arms (11, 12) apart, as will become apparent from the description that follows. The partially semicylindrical longitudinal recesses are therefore inscribed inside a cylinder having the diameter of the rod 18.

The proximal ends (11b, 12b) of the arms (11, 12) have planar faces forming a bearing surface 20 able to receive a distal pressure.

The instrument 10 may for example be manufactured from a material selected from steels and medical-use plastics.

The placement of a clip 1 of FIG. 1, for example in order to bring two fragments of bone closer together using the instrument 10 of FIG. 2A, will now be described with reference to FIGS. 3-12.

The clip 1 at rest is in its closed configuration of FIG. 1.

The instrument 10 is in its position shown in FIG. 2A, with the rod 18 separate, not yet fitted onto the tongs 10a of the instrument 10. The tongs 10a are themselves in an unstressed position, which means to say that the arms (11, 12) are parallel to one another as shown in FIGS. 2A and 2B. Using two fingers, the surgeon applies pressure to the distal ends (11a, 12a) of the two arms (11, 12) of the tongs 10a in order to bring these closer together and bring them into contact with one another, the two arms then making a non-zero angle between them. In such a squeezed-together configuration of the two arms, the dimension of the transverse slot 13 in the transverse direction is slightly less than the length of the crossbar 4 of the clip 1. The surgeon can therefore take hold of the clip 1 and easily load the crossbar 4 into the transverse slot 13 of the front face of the instrument 10, as shown in FIG. 3. The proximal portions of the lateral legs (2, 3) of the clip 1 naturally become housed in the longitudinal slots 14 of the instrument 10. The dimension of the stop-forming structure 15, in the transverse direction, with the lugs (15a, 15b) adjacent to and in contact with one another, is also slightly less than the distance separating the respective proximal portions of the two lateral legs (2, 3) of the clip 1 at rest. As is also apparent from FIG. 3, the stop-forming structure 15 is thus arranged inside the U formed by the clip 1, in the proximal region thereof. The surgeon then releases the pressure he was applying to the two arms (11, 12) of the tongs 10a. The two legs (11, 12) then part very slightly until the structure 15 comes into abutment against the respective proximal portions of said lateral legs (2, 3).

Finally, the distal ends (2a, 3a) of the lateral legs of the clip 1 project out of the instrument 10 in the distal direction. Thus, the proximal region lb of the clip 1 is in the housing formed by the transverse 13 and longitudinal 14 slots. Moreover, the proximal face 13c and the distal face 13d of the transverse slot 13 form blocking means preventing translational movement of the crossbar 4 in the proximal and distal directions. The longitudinal slots 14 themselves form rotation-proofing means preventing the clip 1 from rotating about its crossbar 4. In this position, the clip 1 is still at rest.

The surgeon then takes hold of the rod 18 via its projection 19a for grasping and introduces it via its distal end, using the flat 19b, into the axial hole 17 along the longitudinal axis A in the distal direction as shown in FIG. 4. As it progresses in the distal direction, the distal end of the rod 18 enters the two partially semicylindrical longitudinal recesses (11d, 12d) and causes the spreading of the two distal ends of the arms (11, 12), as shown in FIG. 5. The projection 19a for grasping comes into abutment with the joining bridge 30 whereas the flat 19b comes to bear laterally on the crossbar 4 of the clip 1. The flat 19b thus contributes to preventing the clip 1 from rotating about its crossbar 4 at the time of implantation of said clip 1. The dimension of the stop-forming structure 15 in the transverse direction is increased by the diameter of the rod 18 minus the partially semicylindrical recesses (11d, 12d) and the stop 15 pushes laterally against the respective proximal portions of the lateral legs (2, 3), causing the distal ends (2a, 3a) of these legs to spread. The clip 1 is then under stress loading, in its open configuration. The tongs 10a for their part revert to a configuration identical to the configuration they adopted when not under any stress loading. Thus, once the rod 18 has been inserted, the two arms (11, 12) are once again mutually parallel.

The surgeon can then impact the clip 1, inserting the lateral legs (2, 3) via their distal ends (2a, 3a) into holes 23 made beforehand in the fragments of bone (21, 22) to be pulled together, as shown in FIG. 6. The surgeon applies distal pressure using a surgical hammer (not shown) to the bearing surface 20 of the tongs 10a of the instrument 10 in the direction of the arrow F and inserts the clip 1 approximately to mid-way up the lateral legs (2, 3), as shown in FIG. 7.

The surgeon then removes the rod 18, as shown in FIG. 8. He then uses his fingers to apply pressure to the two arms (11, 12) of the tongs 10a. In so doing, he brings the two lugs (15a, 15b) back into contact with one another and can thus easily disengage the tongs 10a from the proximal region of the clip 1. The clip 1 is half inserted into the fragments of bone, as shown in FIG. 9.

The surgeon then engages the crossbar 4 of the clip 1 in the transverse groove 16 of the distal face of the tongs 10a of the instrument 10, as shown in FIG. 10. Once again, using a surgical hammer (not shown), he applies distal pressure to the bearing surface of the tongs 10a, thus bringing about full insertion of the clip 1 into the fragments of bone (21, 22) as shown in FIG. 11.

The surgeon then removes the tongs 10a, leaving the clip 1 fully inserted into the fragments of bone, as shown in FIG. 12. In this configuration of the clip 1, the two lateral legs, seeking to spring back into their rest position, each apply a return force tending to pull the two fragments of bone (21, 22) closer together.

Reference is made to FIGS. 13 and 14 which depict an instrument 100 according to the invention comprising tongs 10a identical to those of the instrument 10 of FIGS. 1-12 and a removable insert in the form of a rod 24. The references denoting elements of the tongs 10a of FIGS. 1-12 have been kept. The rod 24 is equipped at its proximal end with a knob 25 for grasping. At its distal end, the rod 24 is equipped with an axial slit 26 defining two mutually opposed flats 27. The width of the slit 26, or in other words the distance between the two flats 27, is slightly greater than the width of the crossbar 4 of the clip 1. As with the rod 18 in FIGS. 1-12, the rod 24 is dimensioned in such a way as to be able to pass through the axial hole 17 in the joining bridge 30 of the tongs 10a.

The two flats 27 defined by the axial slit 26 of the rod 24 contribute to keeping the clip 1 in the plane of the tongs 10a, or in other words contribute to preventing the clip 1 from rotating about its crossbar 4 at the time that said clip 1 is being impacted.

Thus, when the clip 1 is mounted on the tongs 10a as explained hereinabove in the case of FIG. 3, the surgeon takes hold of the rod 24 via the knob 25 for grasping and introduces the distal end of the rod 24 into the axial hole 17 along the longitudinal axis A in the distal direction, just as he did with the rod 18 in FIG. 4. During its distal progression, the distal end of the rod 24 engages in the two partially semicylindrical longitudinal recesses (11d, 12d) and causes the two distal ends of the arms (11, 12) to spread, as shown in respect of the rod 18 in FIG. 5. The knob 25 for grasping comes into abutment with the joining bridge 30. The axial slit 26 fits over the crossbar of the clip 1 and each flat 27 comes to bear laterally against the crossbar 4 of the clip 1 on each side of said crossbar 4, as shown in FIG. 14 (just one of the two flats 27 being visible in FIG. 14). The flats 27 thus contribute to preventing the clip 1 from rotating about its crossbar 4 at the time of implantation of said clip 1. The dimension in the transverse direction of the stop-forming structure 15 is increased by the diameter of the rod 24 minus the partially semicylindrical recesses (11d, 12d) and the stop 15 pushes laterally against the respective proximal portions of the lateral legs (2, 3), causing the distal ends (2a, 3a) of these legs to spread, as shown in FIG. 14. The clip 1 is then under stress loading, in its open configuration. The tongs 10a themselves spring back to a configuration identical to the configuration they adopted when under no stress loading. Thus, once the rod 24 has been inserted, the two arms (11, 12) are once again mutually parallel.

The surgeon can then impact the clip 1 in the way described in FIGS. 6-12.

The instrument according to the invention allows an osteosynthesis clip that is U shaped at rest to be loaded, brought into its stress-loaded configuration, then impacted into fragments of bone to be pulled together in a particularly easy manner. In particular, the instrument according to the invention does not require the use of sophisticated additional tools and can be manipulated merely using the fingers.

1. A surgical instrument for implanting a U-shaped osteosynthesis clip having two lateral legs joined together at their respective proximal ends by a crossbar, said crossbar and the proximal portions of said two lateral legs forming a proximal region of said clip, said instrument comprising:
at least one housing intended to temporarily receive said proximal region of said clip, the distal ends of said two lateral legs protruding out of said instrument,
at least blocking means able to prevent translational movement of said crossbar in the proximal and distal directions,
at least rotation-proofing means preventing the clip from rotating about its crossbar,
at least spreader means able to collaborate with said proximal region of said clip in order to increase the distance between the distal ends of the two lateral legs, said spreader means comprising a stop-forming structure located inside the U formed by the clip, in the proximal region thereof, said structure bearing against the proximal portion of each of said lateral legs, said structure being able to increase in size in the transverse direction in order to spread said distal ends of the two lateral legs apart, said structure comprising two adjacent lugs able to be moved relative to one another in the transverse direction, and an insert able to be accommodated temporarily between said two lugs,
said instrument comprising at least one pair of tongs comprising two arms joined together at their proximal ends by a joining bridge, the two arms being parallel when not subjected to any stress loading, said two arms being able to be brought closer together and to form between them a non-zero angle under the effect of pressure applied to each of them toward the other, each of said two lugs being situated on one of said two arms, facing one another,
wherein said insert is a rod the proximal end of which passes through the center of said joining bridge and the distal end of which is accommodated between said two lugs.

2. The instrument as claimed in claim 1, wherein at least part of said spreader means is removable.

3. The instrument as claimed in claim 1, wherein the insert is removable and constitutes a separate piece of said tongs.

4. The instrument as claimed in claim 1, wherein it further comprises, on a distal face, a transverse groove able to accommodate said crossbar.

5. The instrument as claimed in claim 1, wherein it further comprises, on a proximal face, a bearing surface able to receive distal pressure.

6. The instrument as claimed in claim 1, wherein said housing comprises an open groove formed on one face of said instrument.

7. The instrument as claimed in claim 1, wherein said blocking means comprise a transverse slot comprising a proximal face and a distal face, said transverse slot being able to receive the crossbar of the clip.

8. The instrument as claimed in claim 1, wherein said rotation proofing means comprise two longitudinal slots able to receive the proximal portions of the lateral legs of the clip.

9. The instrument as claimed in claim 1, wherein it comprises information means indicating the center distance of the clip intended to be received in said instrument when this clip is under stress loading.

10. The instrument as claimed in claim 9, wherein said information means comprise two transverse holes formed in said instrument, the center distance between said two holes corresponding to the center distance of the clip under stress loading.

11. The instrument as claimed in claim 1, wherein said rotation proofing means comprise at least one flat situated at one distal end of said rod, said at least one flat coming to bear against said crossbar when the proximal region of said clip is received in said housing and the distal end of said rod is accommodated between said two lugs.

12. The instrument as claimed in claim 11, wherein said rotation proofing means comprise two flats situated at one distal end of said rod, each flat coming to bear against said crossbar on each side thereof when the proximal region of said clip is received in said housing and the distal end of said rod is accommodated between said two lugs.

13. A kit comprising an instrument as claimed in claim 1 and at least one U-shaped osteosynthesis clip having two lateral legs joined together at their respective proximal ends by a crossbar.
